(19) United States
(12) Patent Application Publication
(10) Pub. No.: US 2024/0052284 A1
(43) Pub. Date: Feb. 15, 2024

(54) DEVICE FOR PARTICLES HANDLING, WASHING, TRANSFECTION THROUGH ACOUSTOPHORETIC INDUCED MIGRATION

(71) Applicant: AENITIS TECHNOLOGIES, Mitry-Mpry (FR)

(72) Inventors: Jérémie GACHELIN, Arcueil (FR); Emmanuel VINCENT, Mitry-Mory (FR)

(73) Assignee: AENITIS TECHNOLOGIES, Mitry-Mory (FR)

(21) Appl. No.: 17/766,339

(22) PCT Filed: Oct. 16, 2020
(86) PCT No.: PCT/EP2020/079222

§ 371 Date: Apr. 4, 2022

(30) Foreign Application Priority Data
Oct. 18, 2019 (EP) 19306360.9
Oct. 18, 2019 (EP) 19306361.7

(51) Int. Cl. C12M 1/42; C12M 1/00; B01L 3/00; C12M 1/34

(52) U.S. Cl. CPC C12M 35/04; C12M 47/04; B01L 3/502761; C12M 41/36; B01L 2300/0663; B01L 2400/0436; B01L 2300/18; B01L 2400/0439; B01L 2200/0652; B01L 2300/0864

(57) ABSTRACT

A device for separating and/or isolating and/or washing target particles from a particles suspension. The device includes at least two inlets, at least two outlets, a container having a longitudinal axis and a chamber for fluid flow, being configured to be associated with a transducer, at least one transducer configured to generate bulk acoustic waves within the chamber, and at least one flow rate sensor configured to measure the flow rate of the fluid in the chamber. The inlets are located on one end of the container and the outlets are located on the other end along the longitudinal axis. The first and second inlet are each located on either side of the longitudinal axis, the first inlet and the second outlet are each located on either side of the longitudinal axis, and the second inlet and the first outlet are each located on either side of the longitudinal axis.

### FIELD OF INVENTION

The present invention relates to a device for separating and/or isolating and/or washing particles from a suspension by means of an acoustic force field within a channel, notably a microchannel.

### BACKGROUND OF INVENTION

Acoustophoresis can be used to handle and sort particles by means of an acoustic force. In acoustophoresis conventional techniques, known in the prior art, at least one acoustic pressure node is created at a given position along a dimension (length, width or thickness) of a channel by creating a resonance condition for acoustic waves.

WO 2016/201385 A2 describes devices and methods for inspecting, detecting, isolating, monitoring, characterizing, or separating pathogens in blood containing blood cells. The devices include a flow chamber having a solvent inlet, at least one host-fluid inlet, a particulate outlet, at least one residual outlet, and a reflector. The methods include trapping the pathogens in the acoustic standing wave, introducing a solvent into the flow chamber, and removing the pathogens from the device. Devices and methods for inspecting, detecting, isolating, monitoring, characterizing, or separating specialized circulating cells in blood containing blood cells are also disclosed. The devices include a flow chamber having at least one inlet and at least one outlet, and a microscope objective and a cover glass. The methods include driving the transducer to create an acoustic standing wave in the flow chamber and microbubbles in the blood.

However, when not controlled, the flow rate through the chamber may affect the quality of cell separation and lead to poor results. For example, if the concentration of a category of target cells is measured such absence of control of the flow rate may be detrimental.

The invention intends more particularly to remedy to prior art disadvantages by presenting a method and a device, allowing to separate and/or isolate target particles from a suspension and determining a flow rate and/or a concentration of particles of a fluid flowing in a chamber for optimizing the fluid flow, while being easy to use and implement, without the drawbacks of the prior art.

### SUMMARY

The object of the present invention relates to a device for separating and/or isolating and/or washing target particles from a particles suspension, said device comprising:
at least two inlets, wherein at least one first inlet is configured to receive a particles suspension, and a second inlet is configured to receive a buffer solution;
at least two outlets, wherein at least one first outlet is configured to evacuate suspension depleted of target particles, and a second outlet is configured to evacuate separated and/or isolated and/or washed target particles;
a container having a longitudinal axis, comprising a chamber for fluid flow, said chamber being configured to be associated with a transducer;
at least one transducer configured to generate bulk acoustic waves within the chamber, said transducer being located between the at least one first inlet and the at least one first outlet;
at least one flow rate sensor configured to measure the flow rate of the fluid in the chamber;
wherein at least two inlets are located on one end of the container and at least two outlets are located on the other end along longitudinal axis of the container;
wherein the at least one first inlet and the second inlet are each located on either side of longitudinal axis of the container,
wherein the at least one first inlet and the second outlet are each located on either side of longitudinal axis of the container, and
wherein the second inlet and the at least one first outlet are each located on either side of longitudinal axis of the container.

In one embodiment, the at least one transducer is configured to emit stationary bulk acoustic waves. In one embodiment, the bulk acoustic waves are emitted in a direction perpendicular to the longitudinal axis of the container. In one embodiment, the chamber comprises internal walls made of a material having an acoustic impedance superior to the acoustic impedance of the fluid flowing in said chamber. In one embodiment, the device further comprises a coupling element to ensure a temporary coupling between a wall of the chamber and the transducer. In one embodiment, the device further comprises at least one pressure sensor. In one embodiment, the device further comprises at least one concentration sensor configured to measure the concentration of target particles, said concentration sensor being connected to the at least one first inlet and/or at least one first outlet and/or second inlet and/or second outlet. In one embodiment, the device comprises a third inlet configured to receive a second particles suspension and a third outlet configured to evacuate a suspension depleted of target particles, said third inlet and third outlet being symmetrical to a first inlet and a first outlet with respect to the longitudinal axis. In one embodiment, the at least one first inlet has a longitudinal axis perpendicular to the longitudinal axis of the container. In one embodiment, the at least one first outlet has a longitudinal axis perpendicular to the longitudinal axis of the container. In one embodiment, the device further comprises an electronic control unit configured to recover data from the pressure sensor, the concentration sensor and/or the flow rate sensor. In one embodiment, the electronic control unit is configured to monitor the flow rate of the fluid in the chamber on the basis of the recovered data from the pressure sensor, the concentration sensor and/or the flow rate sensor.

The present invention also relates to a method for separating and/or isolating and/or washing target particles from a particles suspension by means of a device according to the invention, said method comprising the steps of:
introducing a particles suspension in the chamber via the at least one first inlet;
simultaneously introducing a buffer solution in the chamber via the second inlet;
activating the at least one transducer to generate bulk acoustic waves in the chamber for separating and/or isolating and/or washing the target particles from the suspension;
evacuating a suspension depleted of target particles from the chamber at the at least one first outlet;
collecting target particles deflected from the suspension by the bulk acoustic waves at the second outlet; and
measuring at least one parameter at the at least one first inlet and/or at least one first outlet and/or second inlet and/or second outlet with at least one sensor configured to measure said at least one parameter.

In one embodiment, the at least one parameter is the pressure and/or concentration of target particles and/or flow rate, wherein the amplitude and the frequency of the bulk acoustic waves are modified as a function of said parameter. In one embodiment, the suspension evacuated from the at least one first outlet comprises at least 75% less target particles than the suspension provided at the at least one first inlet.

The present invention also relates to a use of a device according to the invention to put into contact target particles from a particles suspension with a second type of particles comprised in a buffer solution or another fluid by deflecting the target particles to said buffer solution or said other fluid.

### Definitions

In the present invention, the following terms have the following meanings:
“Active zone”: part of the device facing the transducer.
“Buffer” refers to a fluid in which deflected target particles will be deviated when the device is activated. The buffer may be functionalized through the addition of chemical compounds and/or biological vectors, considered as particles, such as viruses, bacteria, DNA, RNA, plasmids, proteins designed for example to react with or transfect deflected target particles. The buffer may be CPD, SSP+, RPMI medium, LB, HypoThermoSol®, UW solution, HEPES, PBS, CMRL medium or DMEM.
“CMRL”: Connaught Medical Research Laboratories.
“CPD”: Citrate-Phosphate-Dextrose.
“Deflected particles” refers to particles which are deviated from a suspension by acoustic waves emission.
“Depleted fluid” refers to a fluid from which particles have been deflected to go to the enriched fluid when such device is activated.
“Depleted suspension” refers to a suspension from which particles have been deflected to go to the enriched fluid when the device is activated.
“Device activation” refers to acoustic waves emission by the transducer.
“Disposable device” refers to a device designed to be thrown away after a single use.
“DMEM”: Dulbecco's Modified Eagle Medium.
“DMSO”: Dimethyl sulfoxide
“Enriched buffer” refers to a buffer in which the deflected target particles have been oriented at the activation of the device.
“Enriched fluid” refers to a fluid towards which the deflected target particles go, leaving the depleted fluid, when the device is activated.
“Fluid” refers to a liquid or gas which may or may not contain particles (e.g. liquid water).
“HEPES”: 4-(2-HydroxyEthyl)-1-PiperazineEthaneSulfonic acid.
“HypoThermoSol”: Trademark.
“Inlets zone”: part of the device where fluids are injected.
“LB”: Lysogeny Broth.
“Methylcellulose” refers to a chemical compound derived from cellulose.
“Non-deflected particles” refers to particles which are not deviated from a suspension by acoustic waves emission.
“Non-invasive sensor” refers to a sensor which do not interfere with the measured system in a sensible way (e.g.: transit-time flowmeter, echograph, spectrophotometer, Doppler, etc.).
“Opposite wall”: in the active area, wall farthest to and parallel to the main transducer.
“Outlets zone”: part of the device where fluids are extracted.
“Particle” refers to a self-delimited undissolved element comprised in a fluid (e.g. cell, cell clusters, sand, droplet, bubble).
“Particle washing”: action to deviate a particle form a first fluid toward a second fluid.
“Particles suspension” refers to a suspension comprising at least one population of particles, one population being defined by a common size, composition or property. The terms “particles suspension” and “suspension” are used interchangeably herein.
“PBS”: Phosphate-Buffered Saline.
“Pump” refers to a sub-device configured to generate a flow in the device.
“RPMI”: Roswell Park Memorial Institute.
“Sensor” refers to a sub-device configured to perform measurements on a system (e.g.: turbine flowmeter, manometer, endoscope, etc.).
“SSP+”: Storage Solution for Platelets.
“Sterile device” refers to a device free from germs or microorganisms.
“Suspension” refers to a fluid containing particles from which said particles may be deviated.
“System” refers to an element or a group of elements on which measurements can be made (e.g.: a tube containing a flowing fluid, a bag of groceries, a crowd).
“Target particles” refers to particles of interest in a suspension. Said target particles are intended to be deflected from the particles suspension to another fluid (e.g. buffer) to be washed, isolated and/or separated.
“Transducer” refers to a sub-device configured to emit an acoustic wave of a defined frequency and power when receiving a corresponding activating signal.
“Transfer wall”: in the active area, wall closest to and parallel to the main transducer.
“UW solution”: University of Wisconsin cold storage solution.

### DETAILED DESCRIPTION

The following detailed description will be better understood when read in conjunction with the drawings. For the purpose of illustrating, the device is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

Features and advantages of the invention will become apparent from the following description of embodiments of a system, this description being given merely by way of example and with reference to the appended drawings.

The object of the present invention relates to a device for separating and/or isolating and/or washing target particles from a particles suspension, said device comprising:
at least two inlets, wherein at least one first inlet is configured to receive a particles suspension, and a second inlet is configured to receive a buffer solution;
at least two outlets, wherein at least one first outlet is configured to evacuate suspension depleted of target particles, and a second outlet is configured to evacuate separated and/or isolated and/or washed target particles;
a container having a longitudinal axis, comprising a chamber for fluid flow, said chamber being configured to be associated with a transducer;
at least one transducer configured to generate bulk acoustic waves within the chamber, said transducer being located between the at least one first inlet and the at least one first outlet;
at least one flow rate sensor configured to measure the flow rate of the fluid in the chamber;
wherein at least two inlets are located on one end of the container and at least two outlets are located on the other end along longitudinal axis of the container;
wherein the at least one first inlet and the second inlet are each located on either side of longitudinal axis of the container,
wherein the at least one first inlet and the second outlet are each located on either side of longitudinal axis of the container, and
wherein the second inlet and the at least one first outlet are each located on either side of longitudinal axis of the container.

The buffer and the particles suspension flow through the chamber according to a laminar flow and also flow through the acoustic wave emitted by the transducer. The application of an acoustic field on target particles within a chamber induces a movement of said target particles, allowing to gather said target particles at a node of said acoustic field. In the invention, an acoustic pressure node is crated in the center of the chamber (depending on the chosen frequency at which the transducer operates) and acoustic radiation forces (ARF) are generated in the chamber. The ARF push the target particles towards the pressure node with a force of up to a hundred times gravity equivalent. The target particles suspended in the suspension will then migrate to the sound pressure node and be deflected from said suspension to the buffer solution. In other words, layers of buffer solution and particles suspension are created in the chamber by their injection at the first and second inlets, and target particles migrate from the suspension layer to the buffer layer upon generation of the acoustic field in the chamber. The suspension layer can be nearest to the transducer (NT) and the buffer layer(s) can be farthest from the transducer (FT), or buffer layer can be nearest to the transducer (NT) and the suspension layer(s) can be farthest from the transducer (FT), depending on the position of the first and second inlets. The particle enriched fluid (i.e. deflected target particles in buffer) flows towards the second outlet, while the fluid depleted in target particles (i.e. depleted suspension) flows towards the first outlet.

The invention relies on the voluntary deflection of target particles from one fluid to another. Thus, target particles collected at the second outlet are not in the same fluid as they were at the first inlet. This leads to a better isolation, separation or washing of said particles. For example, a buffer layer comprising a fluid having a selective permeability for a particular population of particles can be used to ensure that only said population of particles are collected.

This is particularly advantageous as it allows the isolation, washing or separation of target particles within a fluid without any mechanical force, filtration, or centrifugation steps that could damage said target particles, especially if said target particles are fragile like cells.

Washing refers to the action of deflecting one type of target particles from a particles suspension to a chosen fluid, for example deflecting cells from an enzymatic suspension to a non-enzymatic fluid.

Isolation refers to the action of deflecting one type of target particles from a particles suspension. For example, cells are selected and deflected to a central layer in the chamber to be concentrated.

Separating refers to the action of deflecting target particles from a particles suspension comprising at least two different populations of particles (target and non-target), or the action of deflecting at least two types of target particles from a particles suspension in at least two different directions to at least two different outlets.

The at least two inlets are located on one end of the container and at least two outlets are located on the other end along longitudinal axis of the container; said ends being opposite extremities of the container.

The at least one first inlet and the second inlet are each located on either side of longitudinal axis of the container means that said inlets are located opposite from each other according to the longitudinal axis.

The transducer is configured to emit an acoustic wave, said acoustic wave having a defined amplitude and a defined frequency, said transducer being located between the at least one first inlet and the at least one first outlet. The acoustic waves permit to separate and/or isolate and/or wash target particles from a particles suspension as explained here above.

The at least one transducer configured to generate bulk acoustic waves (BAW), i.e. volume acoustic waves propagating in 3 dimensions, i.e. acoustic waves propagating between the transducer and the chamber through the bulk of the coupling material.

Generating bulk acoustic waves in the chamber advantageously allows for treatment (i.e. isolation, separation and/or washing) of big volumes of particles suspension, allows for an efficient and easy to implement temporary coupling between the transducer and the chamber/container.

The at least one transducer does not generate surface acoustic waves (SAW), stationary surface acoustic waves (SSAW), or travelling surface acoustic waves (TSAW). Surface acoustic waves (SAW) refer to acoustic waves propagating between the transducer and the chamber along a surface. Surface acoustic waves (SSAW) refer to SAW oscillating in time but whose peaks do not travel in space. Travelling surface acoustic waves (TSAW) refer to SAW whose peaks do travel in space. The use of SSAW limits the capacity for temporary coupling between the transducer and the chamber, and the volume processed, making it a poor candidate for the production of contamination sensitive suspensions. The use of travelling waves (e.g TSAW) renders the device of the invention ineffective.

According to one embodiment, the transducer is temporary coupled to the chamber (or container). Temporary coupled means removably coupled. This embodiment is particularly advantageous as it allows to use a disposable container with a non-disposable transducer, thus ensuring the sterility of the container, which remains a primordial point in the handling of biological objects.

According to one embodiment, the transducer is a longitudinal resonator which allow for an efficient and easy to implement temporary coupling between the transducer and the chamber.

According to one embodiment, the transducer is a piezoelectric transducer. The transducer may be made of a ceramic, such as lead zircanate titanate, potassium niobate, or sodium tungstate; a crystal, such as quartz, tourmaline, or gallium phosphate; III-V or II-VI semiconductors such as gallium nitride or zinc oxide; polymers such as polyvinylidene fluoride, polyvinylidene chloride, or polyimide; or a mixture thereof.

The flow rate measurement allows to choose and tune the flow rate within the chamber and make sure that the flow of fluid within the chamber is as smooth as possible in order to respect the Poiseuille's law.

In one embodiment, the flow rate sensor is a contactless flow rate sensor, i.e. said sensor does not have any contact with the fluid in the chamber. This is particularly advantageous as a contactless flow rate measurement/monitoring ensures the sterility of the device and the fluid herein.

In one embodiment, the flow rate sensor is located at the second outlet.

Herein, the at least one means for measuring the flow rate of the fluid in the chamber, the at least one pressure measuring means and/or the at least one means for measuring the concentration of target particles are sensors, respectively a flow rate sensor, a pressure sensor and a concentration sensor.

In a specific configuration, the device is configured to be activated by a transducer, said device comprising:
at least one first inlet being a hydraulic inlet, said hydraulic inlet being configured to receive a particles suspension,
at least one first outlet being a hydraulic outlet for evacuating the suspension,
a container having a longitudinal axis A1, comprising a chamber for fluid flow, said chamber presenting a second inlet being a buffer flow inlet, and a second outlet being a buffer flow outlet for evacuating particles, said buffer flow outlet being located at the opposite of the buffer flow inlet,
at least one means for measuring the flow of the fluid,
wherein the buffer flow inlet and the at least one hydraulic inlet are located on one side of the container while the buffer flow outlet for evacuating the deflected target particles and the at least one hydraulic outlet are located on the other side along the axis A1.

According to one embodiment, target particles are selected in the group comprising biological cells, dispersed cells in a dispersion medium, monodisperse or polydisperse cells, blood cells, platelets, red blood cells, Langerhans islets, white blood cells, cancer cells, stem cell, progenitor cells, bacteria, proteins, liposomes, organelles, cell clusters, viruses, vesicles, microparticles, nanoparticles, microbubbles, microbeads, microorganisms, parasites, algae, sand, sediment, dust, antibodies, powders, gametes, parasite eggs, plankton, tissue, fat, pollen, spores, metal particles, or a mixture thereof.

According to one embodiment, target particles have an average size ranging from 1 μm to 500 μm, preferably ranging from 50 μm to 500 μm, more preferably ranging from 1 μm to 50 μm, most preferably from 1 μm to 20 μm.

Particles larger than 500 μm cannot be efficiently manipulated with the device of the invention, as such a size requires to drastically decrease the frequency of the acoustic waves under 1 MHz where acoustic streaming effect appears.

According to one embodiment, the particles suspension comprises one type of target particles, wherein deviated target particles are evacuated from the chamber at the first outlet and the depleted suspension is evacuated from the chamber at the second outlet, resulting in isolated target particles.

According to one embodiment, the particles suspension comprises at least two different types of particles, wherein a first type of particles is evacuated from the chamber at the first outlet and a second type of particles, the target particles, is evacuated from the chamber at the second outlet, resulting in separated particles. In this embodiment, size and type differences between particles may enable said particles to be separated according to the differences in their migration velocity toward the acoustic pressure node generated along the thickness of the chamber. In a specific configuration of this embodiment, acoustic waves with distinct wavelengths are used to separate each group of particles from the fluid, to do so, two transducers are used.

The buffer solution is chosen according to its acoustic and fluid qualities and its compatibility with the target particles to be deflected. The buffer solution is designed so as to remain in the focal plane of the chamber, for example, by adding a solute or a colloidal suspension for reducing the turbulence or thinning it by adding a solvent such as water. The buffer solution is also designed to reduce the uncontrolled exchange of chemical and particulate type with the suspension.

According to one embodiment, the buffer solution may be, but not limited to, CPD, SSP+, RPMI medium, LB, DMSO, Methylcellulose, HEPES, PBS, CMRL medium, DMEM, or a mixture thereof.

According to one embodiment, the buffer solution is configured not to involuntarily alter the target particles that as to be deviated. For example, so as not to involuntarily alter the target particles, unwanted chemical compounds for target particles are avoided, the osmotic force applied to the target particles may be reduced in the buffer solution and/or an adequate pH value range of target particles is retained.

Alternatively, the buffer may be functionalized through the addition of chemical compounds such as antibiotics, antiviral drugs; and/or biological vectors such as viruses, bacteria or plasmids designed for example to react with or transfect deflected target particles, for example to transfect host cells for cell therapy purposes.

In both cases, when the focal plane is on an acoustic node, the buffer solution has an acoustic impedance superior or equal to the acoustic impedance of the suspension. When the focal plane is between two acoustic nodes, the buffer solution has an acoustic impedance inferior or equal to the acoustic impedance of the suspension.

According to one embodiment, the at least one first outlet and the second outlet are each located on either side of longitudinal axis (A1) of the container at one end of the chamber, said end being opposite to the end where the inlets are located.

According to one embodiment, the at least one transducer is configured to emit stationary bulk acoustic waves.

According to one embodiment, the at least one transducer emits acoustic waves with a frequency range comprised between 100 kHz and 10 MHz, preferably between 500 kHz and 5 MHz. The frequency of the emitted waves is chosen depending on the target particles to be moved in the suspension, in particular said frequency is selected based on the particle size to be deflected. For example, the smaller the particle, the higher the frequency. In most embodiments, the frequency is higher than 1 MHz allowing advantageously to reduce the impact of acoustic streaming.

In one embodiment, the wavelength of the acoustic waves is greater than the average size of the target particles to be separated, preferably greater than or equal to ten times this average size.

According to one embodiment, the at least one transducer is positioned at the center of the lower face of the chamber. Alternatively, the at least one transducer may be positioned on the upper face of the chamber or all along the lower and/or upper face of the chamber.

According to one embodiment, the at least one transducer is external to the chamber.

An acoustic force is related to the emitted acoustic waves and produces a focal plane in the chamber. According to one embodiment, the focal plane is a plane inside the chamber perpendicular to the axis A2a. Depending on the nature of the target particles to be moved, the focal plane may be on the nodes or between two nodes of the acoustic pressure in the chamber.

According to one embodiment, the bulk acoustic waves are emitted in a direction perpendicular to the longitudinal axis of the container. In a specific configuration of this embodiment, the acoustic waves have an incident angle to the longitudinal axis of the chamber ranging from 85° to 95°, e.g. from 89° to 91°. In a more specific configuration of this embodiment, the acoustic waves have an incident angle to the longitudinal axis of the chamber of substantially 90°.

In one embodiment, the transducer generates an acoustic force field over the thickness, not over the width of the chamber. This embodiment is particularly advantageous as it allows the formation of a layer of target particles.

According to one embodiment, the device comprises a plurality of transducers, preferably the device comprises 2, 3, or 4 transducers. This embodiment is advantageous as it allows a better power distribution along the chamber.

In a specific configuration of this embodiment, the transducers may be aligned along the upper face of the chamber or distributed on either side of the chamber according to the longitudinal axis A1. In a specific configuration of this embodiment, the acoustic waves emitted by said transducers may be identical or different. The transducers are preferably aligned along the longitudinal axis (A1) of the container.

The use of a plurality of transducers is advantageous when the fluid flows at high velocity or when layers of large target particles are to be generated. In the first case, the flight time under the transducers decreases as the fluid velocity increases. This may require a greater number of transducers to be used in order to achieve focusing. In the second case, in the absence of flow for example, it is possible to use a plurality of transducers to form layers of large target particles.

When a plurality of transducers is used, at least one of them may generate an acoustic wave along the width of the chamber.

According to one embodiment, the acoustic node generated by the acoustic waves is located at the center of the chamber, i.e. at equal distance from both longitudinal walls of said chamber. Longitudinal walls refer to walls extending along the longitudinal axis (A1). In any case, the acoustic node is not at a wall of the chamber. This advantageously prevents target particles from approaching the inner walls of the chamber, especially inner walls on which said cells can adhere.

According to one embodiment, the chamber comprises internal walls made of a material having an acoustic impedance superior to the acoustic impedance of the fluid flowing in said chamber.

In a specific configuration of this embodiment, the chamber or the internal walls of said chamber are made of a material selected among the group of organic polymers or inorganic polymers; metals; gels, such as for example hydrogel; glass, such as for example fused quartz, pyrex; crystals, such as for example silicon; ceramics, such as for example silicon carbide; resins; derivatives thereof, or a mixture thereof. The material of the chamber or internal walls thereof should be easy to sterilize after use.

Examples of organic polymers include but are not limited to: poly(methyl methacrylate) (PMMA) polyurethane, silicone, polyethylene, polymethylpentene, polystyrene, polycarbonate, polydimethylsiloxane, medical grade polymers, or disposable plastic.

Examples of metals include but are not limited to: steel or stainless steel. Advantageously, stainless steel allows a better transmission of acoustic energy to the fluid in the chamber. Furthermore, a steel wall can play the role of reflector in the device.

Advantageously, PMMA has an acoustic impedance twice as high as that of fluids, stainless steel has an acoustic impedance 40 times higher than that of fluids. Furthermore, PMMA is advantageously compatible with all cells, is optically transparent, i.e. allows to see the interior of the chamber, thus the fluids herein.

According to one embodiment, the device further comprises a coupling element to ensure a temporary coupling between a wall of the chamber and the transducer. Thus the transducer is not glued to the container using, for example an epoxy resin. The coupling element allows for a much more efficient transmission of the acoustic energy between the transducer and the container.

Examples of coupling elements include but are not limited to: a liquid or a gel, such as for example water, hydrogel, vegetable oil, mineral oil, grease, polymer, derivative thereof, or mixture thereof. The coupling element should be as thin as possible, homogeneous, i.e. without any bubbles or microbubbles within said coupling element or on contact surfaces.

The device of the invention does not comprise a reflector configured to reflect the acoustic waves in the chamber. The device of the invention uses a layer of free air at the outside of the chamber as a potential reflector.

According to one embodiment, the device further comprises at least one pressure sensor, i.e. pressure measuring means.

According to one embodiment, the device further comprises at least one concentration sensor (also called herein means for measuring the concentration of target particles) configured to measure the concentration of target particles, said concentration sensor being connected to the at least one first inlet and/or at least one first outlet and/or second inlet and/or second outlet.

According to one embodiment, the at least one means for measuring the flow rate of the fluid in the chamber is a sensor configured to measure the flow rate of the fluid in said chamber. In a specific configuration of this embodiment, the sensor is connected to the at least one first inlet. In this embodiment, the flow rate of the particles suspension is measured by the sensor after being injected in the chamber at the first inlet.

In one embodiment, the sensor may be connected to the first inlet and/or the second inlet and/or the second outlet.

In a preferred configuration of this embodiment, the flow rate sensor, the pressure sensor, and/or the concentration sensor is located (or connected) at the second outlet.

According to one embodiment, the device comprises a plurality of sensors such as for example a flow sensor, a flow rate sensor, a pressure sensor, a sensor for measuring the volumetric concentration of target particles in a fluid or the like.

According to one embodiment, several types of sensors may be used in the different inlets and outlets.

According to one embodiment, each sensor may be connected to one pump.

According to one embodiment, the device comprises a plurality of containers, each container being connected to one sensor, said sensor being between two successive containers and the pumps. In another embodiment, each container may be connected to several sensors, said sensors being connected to several pumps.

Advantageously, the sensor is a non-invasive measuring means, such as a doppler, a transit time flow meter, or a spectrophotometer. In an alternative embodiment, the sensor may be an invasive measuring means.

According to one embodiment, the device further comprises at least one pressure measuring means. Advantageously, the fluid flow, especially the flow rate, in the chamber can be monitored according to the pressure measured.

According to one embodiment, the device further comprises at least one means for measuring the concentration of target particles, said means for measuring the concentration being connected to the at least one first inlet and/or at least one first outlet and/or second inlet and/or second outlet. Advantageously, this embodiment allows a comparison of the concentration of target particles in a suspension and/or buffer solution introduced in the chamber at the at least one first inlet and/or second inlet, and the suspension and/or buffer solution evacuated from said chamber at the at least one first outlet and/or second outlet.

According to one embodiment, the at least one first inlet has a longitudinal axis perpendicular to the longitudinal axis A1 of the container. This configuration allows an easier manufacturing of the chamber, in addition, the assembly is also easier with this configuration. Furthermore, it also allows flexibility regarding the flow rates without having to change the device dimensions.

According to one embodiment, the at least one first outlet has a longitudinal axis perpendicular to the longitudinal axis A1 of the container. This configuration allows an easier manufacturing of the chamber, in addition, the assembly is also easier with this configuration.

In a combination of the previous two embodiments, both the at least one first inlet and at least one first outlet have a longitudinal axis perpendicular to the longitudinal axis A1 of the container.

In an alternative embodiment, the first inlet and the first outlet may be tilted in any directions with regards to the longitudinal axis A1 of the chamber. The first inlet and the first outlet may be arranged parallel to one another. In another embodiment, when the first inlet and the first outlet are not parallel, they are symmetrical with respect to the vertical axis A2a of the transducer.

According to one embodiment, the second inlet and/or the second outlet has a longitudinal axis parallel to the longitudinal axis A1 of the container, i.e. the second inlet and/or the second inlet extends in the same direction than the longitudinal axis A1 of the container, i.e. the second inlet and/or the second inlet is aligned with the longitudinal axis A1 of the container.

In an alternative configuration, the second inlet and/or the second outlet are tilted with respect to the longitudinal axis A1 of the container.

According to one embodiment, the device comprises a third inlet configured to receive a second particles suspension and a third outlet configured to evacuate a suspension depleted of target particles, said third inlet and third outlet being symmetrical to a first inlet and a first outlet with respect to the longitudinal axis. In this embodiment, the third symmetrical inlet and outlet permit to have a higher flow rate and avoid target particles from reaching the internal walls of the chamber so as to prevent said target particles from entering into contact with the internal walls of said chamber. This configuration also allows a much easier activation of resonance.

In this embodiment, the suspension layer can be central with the buffer layer(s) can be lateral, or buffer layer can be central with the suspension layer(s) can be lateral, depending on the position of the inlets. The particle enriched fluid (i.e. deflected target particles in buffer) flows towards the second outlet, while the fluid depleted in target particles (i.e. depleted suspension) flows towards the first and third outlets.

According to one embodiment, the first inlet and/or the second inlet and/or the third inlet (also called second hydraulic inlet) has a longitudinal axis parallel to the longitudinal axis A1 of the container.

According to one embodiment, the first inlet and the third inlet are symmetrical with regards to the longitudinal axis Ala of the chamber. The first outlet and the third outlet are perpendicular to the longitudinal axis Ala of the chamber. The axis of the first outlet and of the third outlet are parallel to the axis A2, preferably, the first outlet and the third outlet have the same axis A2c. In a specific configuration of this embodiment, the first outlet and the second outlet are symmetrical with regards to the longitudinal axis Ala of the chamber.

Herein the longitudinal axis A1 of the container is the longitudinal axis of the chamber.

In one embodiment, the second inlet and the second outlet are aligned with the axis Ala of the chamber. In other embodiments, the second inlet and the second outlet are parallel with this axis A1. The second inlet is positioned at the opposite of the second outlet according to axis A1.

In one embodiment, the inlets and outlets are made integral with the chamber.

According to one embodiment, the inlets and the outlets, especially the first inlet and the first outlet, are positioned at opposite ends (or extremities) of the chamber according to the longitudinal axis A1 of said chamber. Alternatively, the inlets and the outlets, especially the first inlet and the first outlet, may be positioned all along the chamber, on both sides of the transducer.

According to one embodiment, the device further comprises an electronic control unit configured to recover data from the pressure sensor, the concentration sensor and/or the flow rate sensor. In this embodiment, said electric control unit allows managing the pumps and sensors according to the recovered data.

According to one embodiment, the electronic control unit is configured to monitor the flow rate of the fluid in the chamber on the basis of the recovered data from the pressure sensor, the concentration sensor and/or the flow rate sensor.

According to a specific configuration of this embodiment, the electronic control unit recovers parameters from flow rate sensors and adjusts the flow rate of the buffer and/or the suspension pumping at the inlets so as to have a regular flow rate and/or reduce flow disruptions in the chamber. In this configuration, the flow rate measured at the inlets may be equal or different for the buffer and the suspension. For example, if the flow rate sensor measures a flow rate higher at one inlet than the other, the control unit will adjust said flow rate to be the same at both inlets (for example by sending a signal to the pumping means). This is advantageous as it ensures an equal flow rate at the inlets, also prevents fluctuations of flow rate during the isolation/washing/separation of target particles.

According to a specific configuration of this embodiment, the electronic control unit comprises a feedback loop.

According to a specific configuration of this embodiment, the electronic control unit recovers data parameters from a concentration sensor and adjusts the amplitude and/or the frequency of the transducer with regards to the concentration measured by said concentration sensor.

According to one embodiment, the connection between the sensors and the electronic control unit may be wired or wireless.

According to one embodiment, a parameter of pressure and/or concentration and/or flow rate is measured by the at least one measuring means, the amplitude and the frequency of the acoustic waves emitted by the at least one transducer being modified as a function of said parameter by means of an electronic control unit. In this embodiment, the control unit adjusts the flow rate, the frequency and/or the power of the acoustic waves emitted by the transducer depending on the parameter measured by at least one sensor. This ensures an optimal deflection of target particles as, for example, a higher flow rate would require a higher power and potentially a different frequency of the acoustic waves.

According to one embodiment, the device is disposable. The device is usually in contact with fluids that may be contaminated. For hygienic reasons, the device may be for a single-use.

According to one embodiment, the device further comprises at least one pumping means connected to the container. The pumping means are configured to manage the flow of fluid within the device, i.e. within the chamber. In particular, the pumping means able to apply a controlled and constant flow rate at each inlet of the device and/or in the chamber, and advantageously allows to maintain the sterility of the chamber. In this embodiment, the pumping means is a mechanical pump, preferably the pumping means is a peristaltic pump. In a specific configuration, each inlet is connected to one pumping means.

In the case wherein the device comprises a plurality of containers, each container is connected to at least one pumping means.

According to one embodiment, the device comprises at least one non-invasive means for measuring a parameter of pressure and/or concentration and/or flow rate. The use of non-invasive means allows preventing disturbance of the flow within the chamber.

According to one embodiment, the chamber has a rectangular shape, cylindrical shape or other shape. In a preferred configuration of this embodiment, the chamber is a rectangular parallelepiped characterized by a length (along longitudinal axis A1), a width and a thickness, this configuration is particularly advantageous as it is easier to apply an acoustic wave without perturbations to a parallelepiped than any other shape. The chamber can also be called “channel” herein.

According to one embodiment, the chamber is not a microfluidic channel, thus the device of the invention is not a microfluidic device. The use of a non-microfluidic channel allows to significantly and easily increase the flow rate in the container, and so reduce the processing time for a given volume, while keeping the shear rate low enough for the particles not to be damaged during the process, therefore allowing for the use of this device to produce cell therapy treatments.

According to one embodiment, the chamber has a width ranging from 1 mm to 50 mm, preferably from 5 mm to 20 mm, more preferably from 5 mm to 15 mm, said width being the diameter in case of the chamber is a cylinder.

According to one embodiment, the chamber has a thickness ranging from 150 μm to 2 mm, preferably from 300 μm to 780 μm.

According to one embodiment, the chamber has a length ranging from 5 cm to 20 cm, preferably from 5 cm to 15 cm.

In one embodiment, the width of the chamber is, at least at a position along the longitudinal axis at which the acoustic waves are generated, greater than or equal to ten times the average size of the target particles to be separated.

In one embodiment, the acoustic waves have a wavelength λ and the thickness of the chamber is substantially equal to a multiple of λ/4.

According to one embodiment, the device further comprises:
a buffer inlet container configured to be filled with a buffer solution and fluidly connected to the second inlet;
a suspension inlet container configured to be filled with a particles suspension and fluidly connected to the at least one first inlet;
an enriched buffer outlet container fluidly connected to the second outlet; and
a depleted suspension outlet container fluidly connected to the first outlet.

In a specific configuration of this embodiment, said containers are disposable. Disposability ensures a good hygiene, regulatory requirements and saves time.

In a specific configuration of this embodiment, said containers are selected among a bioreactor, a bottle, a bag, a pouch, a vial, a reservoir, a module, or a bottle into which a fluid is transferred, stored or collected.

In a specific configuration of this embodiment, said containers comprise a biocompatible, antimicrobial and/or hypoallergenic material. A biocompatible material is advantageous as it allows contact with a biological fluid. An antimicrobial and/or hypoallergenic material is advantageous as it prevents growth of undesirable microorganisms and/or allergy upon contact with the fluid. Examples of said material comprise but are not limited to: polymer, such as for example an organic polymer or an inorganic polymer; metal, such as for example stainless steel; gel, such as for example hydrogel; glass, such as for example fused quartz, pyrex; crystal, such as for example silicon; ceramic, such as for example silicon carbide; or a mixture thereof. Examples of polymer comprise but are not limited to: polyurethane, silicone, polyethylene, poly(methyl methacrylate) (PMMA), polymethylpentene, polystyrene, polycarbonate, polydimethylsiloxane, or a mixture thereof.

According to one embodiment, the device further comprises a heat transfer system configured to control the temperature of the container and to ensure that said temperature stays below threshold. This embodiment is particularly advantageous at it ensures that the container does not heat up during activation of the transducer (that produces heat), thus that the fluids in the chamber does not heat up. This protects the particles in the container from deterioration due to an excessive heat or an unexpected activation.

In a specific configuration of this embodiment, said threshold is 35° C., preferably 30° C., more preferably 25° C.

In a specific configuration of this embodiment, said heat transfer system is a cooling system such as for example a peltier cooling system or a watercooling system.

The present invention also relates to a method for separating and/or isolating and/or washing target particles from a particles suspension by means of a device according to the invention, said method comprising the steps of:
introducing a particles suspension in the chamber via the at least one first inlet;
simultaneously introducing a buffer solution in the chamber via the second inlet;
activating the at least one transducer to generate bulk acoustic waves in the chamber for separating and/or isolating and/or washing the target particles from the suspension;
evacuating a suspension depleted of target particles from the chamber at the at least one first outlet;
collecting target particles deflected from the suspension by the bulk acoustic waves at the second outlet; and
measuring at least one parameter at the at least one first inlet and/or at least one first outlet and/or second inlet and/or second outlet with at least one means for measuring said at least one parameter.

In other words, in a specific configuration, the method uses a device according to the invention, wherein the method comprises the following steps:
providing the at least one hydraulic inlet (first inlet) with a particles suspension,
providing the buffer flow inlet (second inlet) with a buffer inside the chamber of the container,
activating at least one transducer, said transducer emitting an acoustic wave for separating and/or isolating and/or washing the target particles so as to obtain a depleted suspension,
evacuating the depleted suspension by the at least one hydraulic outlet (first outlet),
evacuating the deflected target particles by the buffer flow outlet (second outlet) of the container,
measuring at least one parameter at the at least one hydraulic inlet and/or outlet and/or at the buffer flow inlet and/or outlet with at least one means for measuring said at least one parameter.

In other words, in another specific configuration, the method uses a device according to the invention, wherein the method comprises the following steps:
providing the at least one hydraulic inlet (second inlet) with a buffer flow,
providing the particles suspension inlet (first inlet) with a particles suspension inside the chamber of the container,
activating at least one transducer, said transducer emitting an acoustic wave for separating and/or isolating and/or washing the target particles so as to obtain a particle depleted suspension,
evacuating the particle depleted suspension by the suspension outlet (first outlet),
evacuating the deviated target particles by the at least one hydraulic outlet (second outlet) of the container,
measuring at least one parameter at the at least one hydraulic inlet and/or outlet and/or at the particles suspension inlet and/or outlet with at least one means.

The method of the invention is a simple and fast method for separating/isolating/washing target particles from a particles suspension.

The method of the invention also enables filterless filtration to be carried out by selective acoustic focusing of the handled particles.

According to one embodiment, the at least one parameter is the pressure and/or concentration of target particles and/or flow rate, wherein the amplitude and the frequency of the bulk acoustic waves are modified as a function of said parameter.

According to one embodiment, the suspension evacuated from the at least one first outlet comprises at least 75% less target particles than the suspension provided at the at least one first inlet, preferably at least 80% less target particles, more preferably at least 90% less target particles.

In a specific configuration of this embodiment, the suspension evacuated from the at least one first outlet comprises target particles. Alternatively, the suspension evacuated from the at least one first outlet is free of target particles.

According to one embodiment, the particles suspension and/or buffer solution are introduced in an inlet according to an injection rate (i.e. the flow rate at the inlets) ranging from 0.1 ml/min to 100 ml/min, preferably from 0.5 ml/min to 10 ml/min. Said injection rate is dependent on the target particles or on the buffer type involved. In a specific configuration of this embodiment, the injection rate of the buffer and the injection rate of the particles suspension are equal. In another specific configuration of this embodiment, the injection rate of the buffer and the injection rate of the particles suspension are different.

According to one embodiment, the particles suspension and/or the buffer solution further comprises an isoacoustic compound configured to render the particles suspension and the buffer solution isoacoustic, i.e. having the same acoustic impedance. Adding an isoacoustic compound to the particles suspension and/or the buffer solution, preferably to the buffer, prevents the fluids entering the chamber from mixing together. Indeed, the aim of the invention is to deflect, i.e. move, only particles from one fluid to another, to do so respective fluids should not be able to mix.

According to one embodiment, the buffer solution further comprises an additive compound for preservation and/or for anticoagulation such as for example SAG-Mannitol (SAGM), PAS III M, citrate-phosphate-dextrose solution (CPD), citrate-acetate-saline based solutions such as T-Sol, Intersol, or SSP+.

In a specific configuration of this embodiment, the isoacoustic compound is selected among the group of, without being restricted to, biocompatible compounds such as dextrose, dextran, glycerol, iodixanol, albumina, cold conservation media such as Hypothermosol or UW Solution®, density gradient colloidal compounds such as Ficoll@, Percoll®, Diacoll®, or a mixture thereof.

According to one embodiment wherein the device comprises a third inlet ad a third outlet, the method comprises the steps of:
introducing a particles suspension in the chamber via the at least one first and third inlets;
simultaneously introducing a buffer solution in the chamber via the second inlet;
activating the at least one transducer to generate bulk acoustic waves in the chamber for separating and/or isolating and/or washing the target particles from the suspension;
evacuating a suspension depleted of target particles from the chamber at the at least one first and third outlets;
collecting target particles deflected from the suspension by the bulk acoustic waves at the second outlet; and
measuring at least one parameter at the at least one first inlet and/or at least one first outlet and/or second inlet and/or second outlet with at least one means for measuring said at least one parameter.

In a specific configuration of this embodiment, the suspension is injected in the first and third inlets in a non-aligned manner with the focal plane of the chamber for extracting the target particles from said suspension, while the buffer solution is injected in the second inlet in an aligned manner.

According to one embodiment, the deflected target particles are collected in a washing medium, such as for example a buffer solution, or a culture medium. This resulting solution is called herein enriched fluid.

According to one embodiment, the method further comprises steps of collecting the enriched fluid (i.e. deflected target particles in buffer solution) and/or collecting the depleted suspension.

According to one embodiment, the method further comprises a supplementary step of treatment of the enriched fluid before optional injection in a subject. Said supplementary step of treatment may include adding a compound to the enriched fluid, depathogenization, filtration, centrifugation, heat/cold treatment, or sampling/testing.

The present invention also relates to the use of the device of the invention for separating and/or isolating and/or washing target particles from a particles suspension.

The present invention also relates to the use of the device of the invention to mix together at least one first type of particles being target particles with at least one second type of particles: a suspension comprising at least one first type of target particles is introduced in the chamber at the first inlet while, simultaneously, a buffer solution comprising at least one second type of particles is introduced in the chamber at the second inlet. Upon generation of acoustic waves in the chamber, the at least one first type of target particles is deviated from the suspension to the buffer solution, resulting in mixing the two types of particles that will be collected at the second outlet while the depleted suspension is collected at the first outlet. The aim is to put into contact target particles from a particles suspension with a second type of particles comprised in a buffer solution or another fluid by deflecting the target particles to said buffer solution (or other fluid).

According to one embodiment, examples of the at least one first type of target particles include, but are not limited to, biological cells, dispersed cells in a dispersion medium, monodisperse or polydisperse cells, blood cells, platelets, Langerhans islets, red blood cells, white blood cells, cancer cells, stem cell, progenitor cells, bacteria, proteins, liposomes, organelles, cell clusters, viruses, vesicles, microparticles, nanoparticles, microbubbles, microbeads, microorganisms, parasites, algae, sand, sediment, dust, antibodies, powders, gametes, parasite eggs, plankton, tissue, fat, pollen, spores, metal particles, or a mixture thereof; and examples of the at least one second type of particles include, but are not limited to, biological cells, dispersed cells in a dispersion medium, monodisperse or polydisperse cells, blood cells, platelets, red blood cells, Langerhans islets, white blood cells, cancer cells, stem cell, progenitor cells, bacteria, proteins, liposomes, organelles, cell clusters, viruses, vesicles, microparticles, nanoparticles, microbubbles, microbeads, microorganisms, parasites, algae, sand, sediment, dust, antibodies, powders, gametes, parasite eggs, plankton, tissue, fat, pollen, spores, metal particles, or a mixture thereof.

In a preferred configuration of this embodiment, the at least one first type of target particles is any biological object with a diameter superior to 1 μm, preferably cells, cell fragments or cells aggregates; and the at least one second type of particles is viral vectors, plasmids, functionalized microbubbles, genetic material.

### DESCRIPTION OF THE DRAWINGS

FIG. 1a is a schematic representation of a device according to one embodiment of the invention.

FIG. 1b is a schematic representation of a device according to one embodiment of the invention comprising three transducers.

FIG. 1c is a schematic representation of a device for isolating target particles from a particles suspension, said device comprising one transducer.

FIG. 1d is a schematic representation of a device for separating target particles from a particles suspension, said device comprising one transducer.

FIG. 2a is a schematic representation of a device according to one embodiment of the invention.

FIG. 2b is a schematic representation of a device for isolating target particles from a particles suspension, said device comprising a first inlet and a third inlet configured to receive a particles suspension, a second inlet configured to receive a buffer solution, and a first outlet and a third outlet configured to evacuate a target particles depleted suspension, a second outlet configured to evacuate deflected target particles in a buffer.

FIG. 2c is a schematic representation of a device for separating target particles from a particles suspension, said device comprising a first inlet and a third inlet configured to receive a particles suspension, a second inlet configured to receive a buffer solution, and a first outlet and a third outlet configured to evacuate a target particles depleted suspension, a second outlet configured to evacuate deflected target particles in a buffer.

FIG. 3 is an overall schematic representation of a device comprising pumps and sensors.

FIG. 4 shows the isolation efficiency obtained for different wave powers using the device of the invention.

FIG. 5 illustrates blood fractionation, i.e. RBC/PLT separation, using the device of the invention.

While various embodiments have been described and illustrated, the detailed description is not to be construed as being limited hereto. Various modifications can be made to the embodiments by those skilled in the art without departing from the true spirit and scope of the disclosure as defined by the claims.

### ILLUSTRATIVE EMBODIMENTS OF THE INVENTION

The following embodiments are not limited to a single application. All the features of each embodiment can be taken into consideration in the other embodiments described.

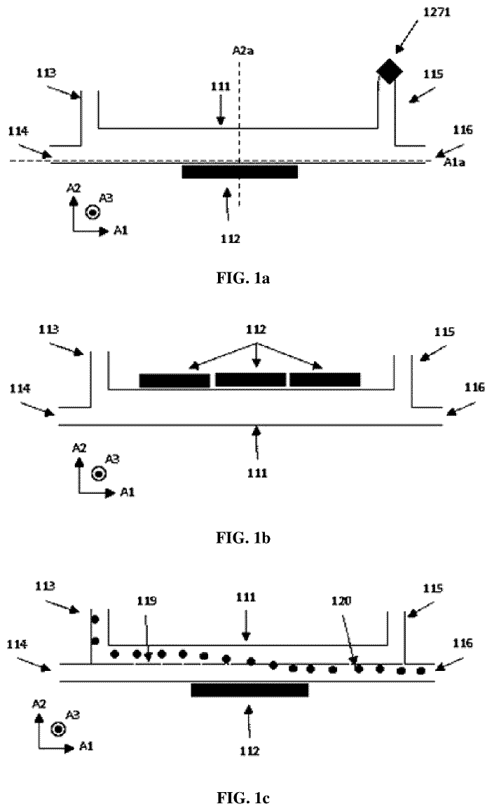

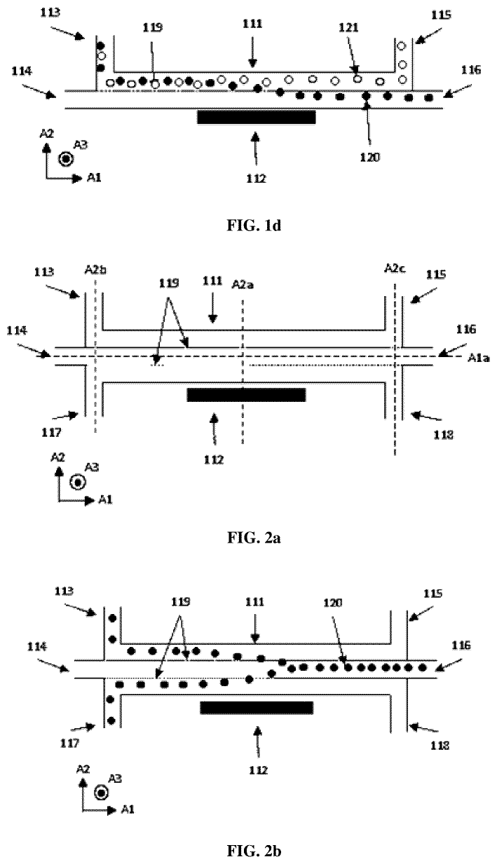

In FIG. 1a, a schematic representation of a device according to the invention is presented.

In a first embodiment, the device comprises a chamber 111 with a first inlet being a hydraulic inlet 113, a second inlet being a buffer flow inlet 114, a first outlet being a hydraulic outlet 115, a second inlet being a buffer flow outlet 116, a sensor 1271 and a transducer 112.

In this embodiment, the first hydraulic inlet 113 and the first hydraulic outlet 115 are perpendicular to the longitudinal axis A1 of the chamber 111. The first hydraulic inlet 113 and the first hydraulic outlet 115 are positioned at opposite ends of the chamber 111.

In this embodiment, a sensor 1271 is connected to the first hydraulic outlet 115. The sensor 1271 is a means for measuring the flow rate of the fluid flowing from the inlet to the first hydraulic outlet 115. In this embodiment, the flow rate of the suspension is measured by the sensor 1271 after being injected in the chamber 111 into the first hydraulic inlet 113.

In an alternative, the device comprises a chamber 111 with a first inlet being a hydraulic inlet 114, a second inlet being a buffer flow inlet 113, a first outlet being a hydraulic outlet 116, a second inlet being a buffer flow outlet 115, a sensor 1271 and a transducer 112.

In this alternative, the first hydraulic inlet 114 and the first hydraulic outlet 116 are perpendicular to the longitudinal axis A1 of the chamber 111. The first hydraulic inlet 114 and the first hydraulic outlet 116 are positioned at opposite ends of the chamber 111.

In this alternative, a sensor 1271 is connected to the first hydraulic outlet 116. The sensor 1271 is a means for measuring the flow rate of the fluid flowing from the inlet to the first hydraulic outlet 116. In this embodiment, the flow rate of the suspension is measured by the sensor 1271 after being injected in the chamber 111 into the first hydraulic inlet 114.

In another embodiment illustrated in FIG. 1b, the device further comprises a plurality of transducers 112.

In another embodiment illustrated in FIG. 1c, a device for isolating target particles from a suspension is presented. The device comprises a chamber 111 with a first inlet being a hydraulic inlet 113, a second inlet being a buffer flow inlet 114, a first outlet being a hydraulic outlet 115, a second inlet being a buffer flow outlet 116 and a transducer 112.

The device is divided into three zones: an inlet zone, an active zone and an outlet zone. In the FIG. 1c, the chamber 111 is represented with delimitations 119, said delimitations are only illustrative to show said layers (suspension and buffer) and should not be understood as physical or mechanical delimitations.

The inlet zone comprises the first inlet 113 and the second inlet 114, the active zone is designed to receive the transducer 112, the outlet zone comprises the first outlet 115 and the second outlet 116. The active zone is designed to optimize sound efficiency in a controlled manner through the selection of materials, layer thicknesses and chamber dimensions. The active zone is divided in several parts, it comprises a transfer wall, a fluid chamber and an opposite wall. The transfer wall corresponds to the lower face of the chamber 111, the fluid chamber corresponds to the area wherein the fluid flows through the chamber 111 from one inlet to one outlet, and the opposite wall corresponds to the upper face of the chamber 111. The opposite wall is located opposite from the transfer wall according to the longitudinal axis A1. In this embodiment, the transfer wall and the opposite wall may have the same dimensions but, in another embodiment, the dimensions may be different.

A particles suspension is injected into the first inlet 113, said particles suspension comprising one type of target particles 120. The suspension is injected in the first inlet 113 according to a non-aligned manner with the focal plane of the chamber 111 for extracting the target particles 120 from the suspension. A buffer solution is simultaneously (i.e. at the same time) injected in the second inlet 114 in an aligned manner for placing the first type of target particles 120.

The buffer and the suspension flow through the chamber 111, therefore flowing through the acoustic waves emitted by the transducer 112. During the transition in the active zone, the target particles 120 are deflected from the suspension to the buffer solution, resulting in a depleted suspension and a particle enriched fluid. The particle enriched fluid 120 flows towards the second outlet 116, while the depleted suspension flows towards the first outlet 115. This embodiment results in target particles isolated from the suspension. In an alternative, the device comprises a chamber 111 with a first inlet being a hydraulic inlet 114, a second inlet being a buffer flow inlet 113, a first outlet being a hydraulic outlet 116, a second inlet being a buffer flow outlet 115 and a transducer 112. A particles suspension is injected into the first inlet 114, said particles suspension comprising one type of target particles 120. The suspension is injected in the first inlet 114 according to a non-aligned manner with the focal plane of the chamber 111 while a buffer solution is simultaneously injected in the second inlet 113 in an aligned manner.

In this alternative, the buffer and the suspension flow through the chamber 111, therefore flowing through the acoustic waves emitted by the transducer 112. During the transition in the active zone, the target particles 120 are deflected from the suspension to the buffer solution, resulting in a depleted suspension and a particle enriched fluid. The particle enriched fluid 120 flows towards the second outlet 115, while the depleted suspension flows towards the first outlet 116. This embodiment results in target particles isolated from the suspension.

In another embodiment illustrated in FIG. 1d, a device for separating target particles from a suspension is presented. The device comprises a chamber 111 with a first inlet being a hydraulic inlet 113, a second inlet being a buffer flow inlet 114, a first outlet being a hydraulic outlet 115, a second inlet being a buffer flow outlet 116 and a transducer 112. In the FIG. 1d, the chamber 111 is designed with delimitations 119, said delimitations are only illustrative (to show suspension layer and buffer layer) and should not be understood as physical or mechanical delimitations.

A suspension is injected into the first inlet 113, the suspension contains two types of particles 120 and 121. At the same time, a buffer solution is injected into the second inlet 114. The buffer solution and the suspension flow through the chamber 111, therefore flowing through the acoustic waves emitted by the transducer 112. During the transition in the active zone, the particles (120, 121) are separated. The first type of target particles 120 flows towards the second outlet 116, since it has been deflected by the acoustic wave, while the second type of particles 121 (that has not been deflected) exits the chamber 111 through the first outlet 115. In this embodiment, the different particles are separated in the suspension.

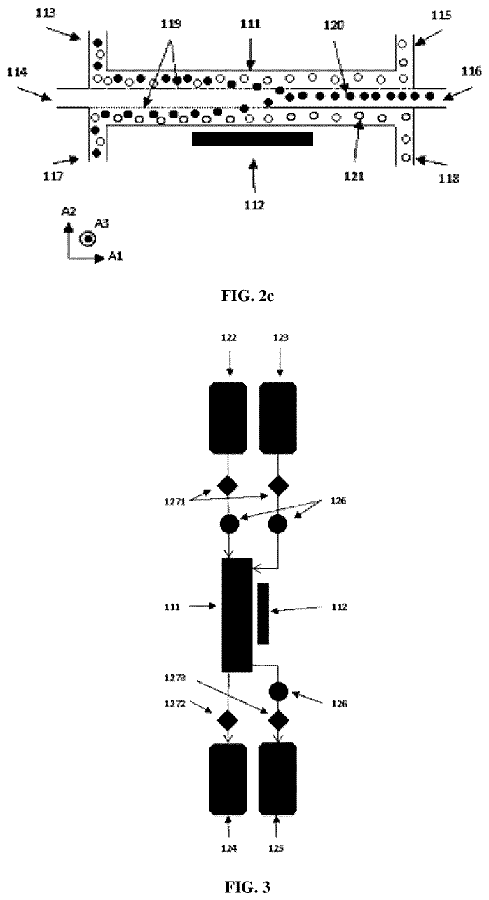

In another embodiment illustrated in FIG. 2a, a device for separating and/or isolating and/or washing target particles from a suspension is presented. The device comprises a chamber 111 with one first inlet 113 (also called first hydraulic inlet), a third inlet 117 (also called second hydraulic inlet), a second inlet 114 (also called buffer flow inlet), a first outlet 115 (also called first hydraulic outlet), a third outlet 118 (also called second hydraulic outlet), a second outlet 116 (also called buffer flow outlet) and a transducer 112. In the FIG. 2a, the chamber 111 is designed with delimitations 119, said delimitations are only illustrative and should not be understood as physical or mechanical delimitations. The first inlet 113 and the third inlet 117 have the same axis A2b. The first and third inlets (113, 117) and the first and third outlets (115,118) are positioned at the opposite ends of the chamber 111. The second inlet 114 and the second outlet 116 are aligned with the axis Ala of the chamber 111. The transducer 112 is positioned at the center of the lower face of the chamber 111.

The device is divided into three zones (delimited by delimitations 119) which are the suspension in the lateral layers and the buffer in the central layer. The inlet zone comprises the inlets (113, 114, 117), the active zone is designed to receive the transducer 112, the outlet zone comprises the outlets (115, 116, 118). The active zone is designed to optimize sound efficiency in a controlled manner through the selection of materials and/or layer thicknesses and/or chamber width. The active zone is divided in several parts, it comprises a transfer wall, a fluid chamber and an opposite wall. The transfer wall corresponds to the lower face of the chamber 111, the fluid chamber corresponds to the area wherein the fluid flows through the chamber 111 and the opposite wall corresponds to the upper face of the chamber 111, opposite from transfer wall.

In another embodiment illustrated in FIG. 2b, a device for isolating target particles from a suspension is presented. The device comprises a chamber 111 with one first inlet 113 (also called first hydraulic inlet), a third inlet 117 (also called second hydraulic inlet), a second inlet 114 (also called buffer flow inlet), a first outlet 115 (also called first hydraulic outlet), a third outlet 118 (also called second hydraulic outlet), a second outlet 116 (also called buffer flow outlet) and a transducer 112. The chamber 111 is designed with delimitations 119, said delimitations are only illustrative and should not be understood as physical or mechanical delimitations.

A suspension comprising a first type of particle type 120 is injected into the first and third inlets (113, 117) according to a non-aligned manner with the focal plane of the chamber 111 for extracting the target particles 120 from the suspension. A buffer solution is injected into the second inlet 114 at the same time in an aligned manner. The injection rate of the buffer and the injection rate of the suspension are equal.

The buffer and the suspension flow inside the chamber 111 and flow through the acoustic waves emitted by the transducer 112. During the transition in the active zone, the target particles 120 are deflected from the suspension into the buffer solution flow, resulting in a depleted suspension and a particle enriched fluid (comprising target particles in the buffer solution). The particle enriched fluid 120 flows to the second outlet 116, while depleted suspension flows to the first and third outlets (115, 118). This embodiment results in target particles isolated from the suspension.

In an alternative embodiment, after flowing through the active zone, the particle enriched fluid 120 flows to the first and third outlets (115, 118) while depleted suspension flows to the second outlet 116.

In another embodiment illustrated in FIG. 2c, a device for separating target particles from a suspension is presented. The device comprises a chamber 111 with one first inlet 113 (also called first hydraulic inlet), a third inlet 117 (also called second hydraulic inlet), a second inlet 114 (also called buffer flow inlet), a first outlet 115 (also called first hydraulic outlet), a third outlet 118 (also called second hydraulic outlet), a second outlet 116 (also called buffer flow outlet) and a transducer 112. The chamber 111 is designed with delimitations 119, said delimitations are only illustrative and should not be understood as physical or mechanical delimitations.

A suspension comprising two types of particles (120, 121) is injected into the first and third inlets (113, 117). At the same time, a buffer solution is injected into the second inlet 114. The buffer solution and the suspension flow inside the chamber 111, therefore flowing through the acoustic waves emitted by the transducer 112. During the transition in the active zone, the two types of particles (120, 121) are separated and each type of deflected target particles flows to an outlet. The first type of target particles 120 flows towards the second outlet 116, due to deflection induced by the acoustic waves, while the second type of particles 121, which are not deflected, exits through the first outlet 115 and the third outlet 118. In this embodiment, the different particles are separated from the suspension and from each other.

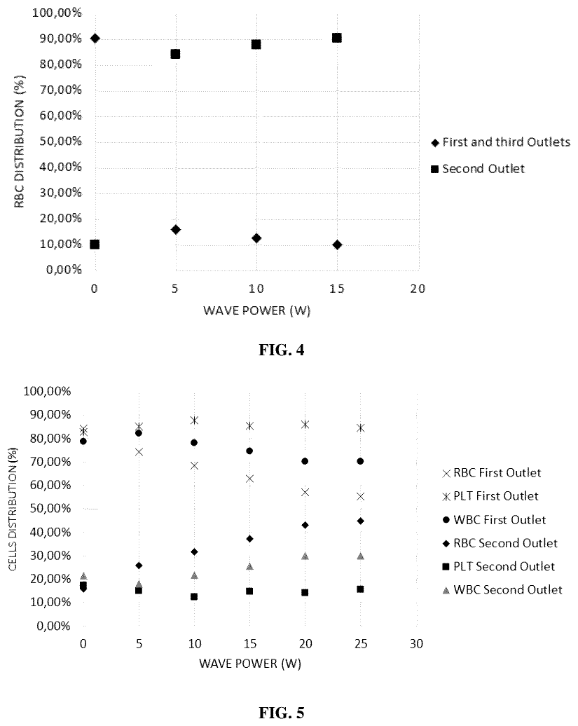

In another embodiment illustrated in FIG. 3, a device with pumps and sensors is presented. All the previous embodiments can be integrated into the schematic representation in FIG. 3. The device comprises a chamber 111, a transducer 112, several pumps 126, several sensors (1271,1272,1273), a buffer inlet container 122, a suspension inlet container 123, an enriched buffer outlet container 124 and a depleted suspension outlet container 125. The buffer inlet container 122 is filled with a buffer solution while the suspension inlet container 123 is filled with a particles suspension. The buffer inlet container 122 and the suspension inlet container 123 are connected to several sensors 1271. Each sensor 1271 may be connected to one pump 126. In this embodiment, each container (122, 123) is connected to one sensor 1271, said sensor 1271 being between the containers (122, 123) and the pumps 126. The pump 126 connected to the buffer inlet container 122 and to the sensor 1271, is also connected to the second inlet 114 of the chamber 111 (presented in FIG. 1a or 2a). The pump 126 connected to the suspension inlet container 123 and to the sensor 1271, is also connected to one of the first and/or third inlets of the chamber 111 (presented in FIG. 1a or 2a).

The first and/or third outlet (115,118) of the chamber 111 (presented in FIG. 1a or 2a) is connected to the depleted suspension outlet container 125 via one pump 126 and one sensor 1273. In this embodiment, the second outlet 116 of the chamber 111 (presented in FIG. 1a or 2a) is connected to the enriched buffer outlet container 124 and only one sensor 1272.

The outlet containers (124,125) may be empty before use or they may contain a liquid such as CPD, SSP+, RPMI medium, LB, DMSO, Methylcellulose, HEPES, PBS, CMRL medium, DMEM, a mixture thereof, or other fluid for preserving the particle depleted fluid and/or the particle enriched buffer inside.

In this embodiment, the suspension contained in the suspension inlet container 123 is injected via a first pump 126 into the chamber 111. At the same time, the buffer contained in the buffer inlet container 122 is injected via a second pump 126 into the chamber 111. The transducer 112 emits acoustic waves at a specific amplitude and a specific frequency when the suspension and the buffer flow inside the chamber 111. After deflection of target particles due to the acoustic waves generated by the transducer 112, the resulting particle enriched fluid flows from the chamber 111 to the enriched buffer outlet container 124. A sensor 1272 is connected to the second outlet 116 of the chamber 111. The depleted suspension flows from the chamber 111 to the depleted suspension outlet container 125. A sensor 1273 is also connected to one of the first and/or third outlet of the chamber 111 with a third pump 126.

In this embodiment, the sensors 1271 are flow rate sensors: the flow rate of the suspension and the buffer are measured by the sensors 1271 before being injected in the chamber 111. The sensor 1272 measures the concentration of the enriched fluid going into the enriched buffer outlet container 124. The sensor 1273 measures the flow rate at the first and third outlets of the chamber 111.

### EXAMPLES

The present invention is further illustrated by the following examples.

### Example 1a: Isolation of Target Particles

Device and Methods

The isolation device comprises an acoustic generator and amplifier connected to a piezoelectric transducer, a heat transfer system to keep the transducer below 25° C. Peristaltic pumps are used to generate a flow in the device (at about 1.5 ml/min).

The isolation device also comprises a container and:
a chamber,
a second inlet (buffer inlet) being the center inlet,
a first inlet (first suspension inlet) and a third inlet (second suspension inlet) located on opposite sides of the container in regards to longitudinal axis (A1),
a first outlet (first depleted suspension outlet) and a third outlet (second depleted suspension outlet) located on opposite sides of the container in regards to longitudinal axis (A1),
a second outlet (enriched fluid outlet) being the center outlet.

The inlets are located on one end of the container and the outlets are located on the other end along longitudinal axis (A1) of the container. The first inlet and the third outlet are each located on either side of longitudinal axis (A1) of the container.

The chamber of the device is designed in PMMA, the tubing is in silicon. The device is coupled to the setup using oil.

The particles suspension processed is human blood diluted in an isotonic solution (down to an hematocrit of 14%). The buffer solution is composed of an isotonic buffer supplemented with Dextran 40 (5%). The suspension is injected in the device at 1.2 ml/min, the buffer at 1.1 ml/min. The enriched fluid outlet is driven at 1.1 ml/min, the depleted outlets are driven at 1.2 ml/min. Once the flow is established in the chamber, a sound wave at 1 MHz is emitted and transferred in the chamber. The outlets are sampled for analysis through a hematology analyzer, determining the concentration in blood cells in the enriched fluid and depleted suspension.

Results

Red Blood Cells (RBC) are transferred from one fluid to the other when the acoustic wave in in effect, resulting in RBC isolation from the original suspension.

FIG. 4 shows the isolation efficiency obtained for different wave powers. An isolation efficiency of 90% is reached at 15W.

### Example 1b: Isolation of Target Particles

Example 1a is reproduced with different types of target particles separated with the same device of example 1a.

Results are reported in Table 1.

TABLE I
Results for isolation of different types of target particles.

| | [target | | [target | | [target | [target |
|---|---|---|---|---|---|---|
| | particles] in | | particles] in | | particles] in | particles] in |
| Target | suspension at | Flow rate at | buffer at | Flow rate at | suspension at | buffer at |
| particles | first inlet | first inlet | second inlet | second inlet | first outlet | second outlet |
| Platelets | 100% | 1.2 ml/min | 0% | 1.1 ml/min | 15% | 85% |
| White Blood | 100% | 1.2 ml/min | 0% | 1.1 ml/min | 10% | 90% |
| Cells | | | | | | |
| Langherans | 100% | 1.3 ml/min | 0% | 1.1 ml/min | 7% | 93% |
| Islets | | | | | | |
| Mesenchymal | 100% | 1.2 ml/min | 0% | 1.1 ml/min | 11% | 89% |
| stem cells | | | | | | |

### Example 2a: Separation of Target Particles

Device and Methods

The separation device is identical to the isolation device used in Example 1a.

The particles suspension processed is human blood diluted in an isotonic solution (down to an hematocrit of 17%). The buffer solution is composed of an isotonic buffer supplemented with Dextran 40 (11%).

The suspension is injected in the device at 1.2 ml/min, the buffer at 1.1 ml/min. The enriched outlet is driven at 1.1 ml/min, the depleted outlets are driven at 1.2 ml/min. Once the flow in the chamber is established, a sound wave at 1 MHz and is emitted and transferred in the chamber. The outlets are sampled for analysis through an hematology analyzer, determining the concentration in blood cells in the enriched fluid and in the depleted suspension.

Results

FIG. 5 shows the results of the experiment.

45% of Red Blood Cells (RBC) are transferred from one fluid (suspension) to the other (buffer solution) when the acoustic wave in in effect, while only 17% of platelets (PLT) are deflected. This method allows for blood fractionation, in particular RBC/PLT separation.

### Example 2b: Separation of Target Particles

Example 2a is reproduced with different types of particles (Particles I and Particles II) separated with the same device of example 2a.

Results are reported in Table 2. Particles I or “target particles” are noted P-I and Particles II are noted P-II. The concentration of particles ([P-X]) at the first inlet and first outlet refers to the concentration of particles in suspension. The concentration of particles ([P-X]) at the second inlet and second outlet refers to the concentration of particles in buffer.

TABLE II
Results for separation of different types of target particles.

| | | [P-I] at | [P-II] at | Flow rate | [P] at | Flow rate | [P-I] at | [P-II] at | [P-I] at | [P-II] at |
|---|---|---|---|---|---|---|---|---|---|---|
| P-I | P-II | 1^{st} inlet | 1^{st} inlet | at 1^{st} inlet | 2^{nd} inlet | at 2^{nd} inlet | 1^{st} outlet | 1^{st} outlet | 2^{nd} outlet | 2^{nd} outlet |
| Langherans | Exocrin | 100% | 100% | 1.2 ml/min | 0% | 1.1 ml/min | 62% | 95% | 38% | 5% |
| Islet | Tissue | | | | | | | | | |
| Mesenchymal | Blood | 100% | 100% | 1.2 ml/min | 0% | 1.1 ml/min | 12% | 48% | 88% | 52% |
| stem cells | cells | | | | | | | | | |

### REFERENCES

111: Chamber
112: Transducer
113: Inlet
114: Inlet
115: Outlet
116: Outlet
117: Third inlet
118: Third outlet
119: delimitations as fluid separation line (solely intended for illustration purposes)
120: First type of particles
121: Second type of particles
122: Buffer inlet container
123: Suspension inlet container
124: Enriched buffer outlet container
125: Depleted suspension outlet container
126: Pumps
1271: Sensor
1272: Sensor
1273: Sensor

1.-15. (canceled)

16. A device for separating and/or isolating and/or washing target particles from a particles suspension, said device comprising:
at least two inlets, wherein at least one first inlet is configured to receive a particles suspension, and a second inlet is configured to receive a buffer solution;
at least two outlets, wherein at least one first outlet is configured to evacuate suspension depleted of target particles, and a second outlet is configured to evacuate separated and/or isolated and/or washed target particles;
a container having a longitudinal axis, comprising a chamber for fluid flow, said chamber being configured to be associated with a transducer;
at least one transducer configured to generate bulk acoustic waves within the chamber, said transducer being located between the at least one first inlet and the at least one first outlet; and
at least one flow rate sensor configured to measure the flow rate of the fluid in the chamber;
wherein at least two inlets are located on one end of the container and at least two outlets are located on the other end along longitudinal axis of the container;
wherein the at least one first inlet and the second inlet are each located on either side of longitudinal axis of the container,
wherein the at least one first inlet and the second outlet are each located on either side of longitudinal axis of the container,
wherein the second inlet and the at least one first outlet are each located on either side of longitudinal axis of the container,
wherein the chamber comprises internal walls made of a material having an acoustic impedance superior to the acoustic impedance of the fluid flowing in said chamber, and
wherein the device further comprises a coupling element to ensure a temporary coupling between a wall of the chamber and the transducer.

17. The device according to claim 16, wherein the bulk acoustic waves are emitted in a direction perpendicular to the longitudinal axis of the container.

18. The device according to claim 16, further comprising at least one pressure sensor.

19. The device according to claim 16, further comprising at least one concentration sensor configured to measure the concentration of target particles, said concentration sensor being connected to the at least one first inlet and/or at least one first outlet and/or second inlet and/or second outlet.

20. The device according to claim 16, wherein the device comprises a third inlet configured to receive a second particles suspension and a third outlet configured to evacuate a suspension depleted of target particles, said third inlet and third outlet being symmetrical to a first inlet and a first outlet with respect to the longitudinal axis.

21. The device according to claim 16, wherein the at least one first inlet has a longitudinal axis perpendicular to the longitudinal axis of the container.

22. The device according to claim 16, wherein the at least one first outlet has a longitudinal axis perpendicular to the longitudinal axis of the container.

23. The device according to claim 16, further comprising an electronic control unit configured to recover data from the pressure sensor, the concentration sensor and/or the flow rate sensor.

24. The device according to claim 23, wherein the electronic control unit is configured to monitor the flow rate of the fluid in the chamber on the basis of the recovered data from the pressure sensor, the concentration sensor and/or the flow rate sensor.

25. A method for separating and/or isolating and/or washing target particles from a particles suspension by means of a device according to claim 16, said method comprising the steps of:
introducing a particles suspension in the chamber via the at least one first inlet;
simultaneously introducing a buffer solution in the chamber via the second inlet;
activating the at least one transducer to generate bulk acoustic waves in the chamber for separating and/or isolating and/or washing the target particles from the suspension;
evacuating a suspension depleted of target particles from the chamber at the at least one first outlet;
collecting target particles deflected from the suspension by the bulk acoustic waves at the second outlet; and
measuring at least one parameter at the at least one first inlet and/or at least one first outlet and/or second inlet and/or second outlet with at least one sensor.

26. The method according to claim 25, wherein the at least one parameter is the pressure and/or concentration of target particles and/or flow rate, wherein the amplitude and the frequency of the bulk acoustic waves are modified as a function of said parameter.

27. The method according to claim 25, wherein the suspension evacuated from the at least one first outlet comprises at least 75% less target particles than the suspension provided at the at least one first inlet.

28. A method for separating and/or isolating and/or washing target particles with the device according to claim 16, comprising contacting target particles from a particles suspension with a second type of particles comprised in a buffer solution or another fluid by deflecting the target particles to said buffer solution or said other fluid.
